(19) United States
(12) Patent Application Publication
(10) Pub. No.: US 2019/0380314 A1
(43) Pub. Date: Dec. 19, 2019

(54) Methods of Genetic Modification of a Cell

(71) Applicant: President and Fellows of Harvard College, Cambridge, MA (US)

(72) Inventors: George M. Church, Brookline, MA (US); Ellen L. Shrock, Brookline, MA (US); Yinan Kan, Cambridge, MA (US)

(73) Assignee: President and Fellows of Harvard College, Cambridge, MA (US)

(21) Appl. No.: 16/484,616

(22) PCT Filed: Feb. 23, 2018
(86) PCT No.: PCT/US18/19313

§ 371 Date: Aug. 8, 2019

Related U.S. Application Data
(60) Provisional application No. 62/462,409, filed on Feb. 23, 2017.

(51) Int. Cl. A01K 67/027; C07K 14/705; C12N 15/113; C12N 15/873

(52) U.S. Cl. CPC A01K 67/0275; C07K 14/705; C12N 15/1138; C12N 2310/20; A01K 2217/075; A01K 2227/108; A01K 2267/025; C12N 15/873

(57) ABSTRACT

The disclosure provides a method of modifying a PERV-A receptor gene in a cell. The method includes introducing into the cell a nucleic acid sequence encoding a Cas9 protein and a nucleic acid sequence encoding a guide RNA, introducing into the cell a donor nucleic acid sequence, wherein the Cas9 protein and the guide RNA are expressed and co-localize at a genomic site near or in the PERV-A receptor gene and the donor nucleic acid sequence replaces the PERV-A receptor gene by homology directed repair (HDR).

### RELATED APPLICATION DATA

This application claims priority to U.S. Provisional Application No. 62/462,409 filed on Feb. 23, 2017, which is hereby incorporated herein by reference in its entirety for all purposes.

### STATEMENT OF GOVERNMENT INTERESTS

This invention was made with government support under HG008525 awarded by National Institutes of Health. The government has certain rights in the invention.

### FIELD

The present invention relates in general to methods of genetic modification of a cell.

### BACKGROUND

The shortage of human organs and tissues for transplantation represents one of the most significant unmet medical needs. In the United States and Europe alone, approximately 200,000 patients await organ transplantation, but only a small fraction receives donor organs (US Government, No Title. U.S. Dep. Heal. Hum. Serv., (available at https://optn.transplant.hrsa.gov/); European Commission, Journalist Workshop on Organ donation and transplantation Recent Facts & Figures., (available at http://ec.europa.eu/health/index_en.htm), hereby incorporated by reference in their entireties). Xenotransplantation, or cross-species transplantation, has the potential to provide an almost unlimited supply of transplant organs for patients with chronic organ failure. However, the clinical use of porcine organs has been hindered, in part, by the potential risk of porcine endogenous retrovirus (PERV) transmission to humans.

PERVs are proviruses in the porcine genome that originated as viral DNA that became integrated in germ line chromosomes during exogenous retroviral infections (See, e.g., Mattiuzzo, G. & Takeuchi, Y. Suboptimal Porcine Endogenous Retrovirus Infection in Non-Human Primate Cells: Implication for Preclinical Xenotransplantation. PLOS ONE 5, e13203 (2010), hereby incorporated by reference in its entirety). Since they became integrated after the evolutionary divergence of the lineages leading to human and pig species, they are not present in the human genome. Most PERVs have become defective over time, but certain intact PERVs have been shown to infect human cells in vitro (See, e.g., Patience, C., Takeuchi, Y. & Weiss, R. A. Infection of human cells by an endogenous retrovirus of pigs. Nat. Med. 3, 282-286 (1997); Yang, L. et al. Genome-wide inactivation of porcine endogenous retroviruses (PERVs). Science 350, 1101-1104 (2015), hereby incorporated by reference in their entireties). These intact PERVs pose a potential risk of zoonosis in pig-to-human xenotransplantation (See, e.g., Denner, J., Tönjes, R. R., Takeuchi, Y., Fishman, J. & Scobie, L. First update of the International Xenotransplantation Association consensus statement on conditions for undertaking clinical trials of porcine islet products in type 1 diabetes-Chapter 5: recipient monitoring and response plan for preventing disease transmission. Xenotransplantation 23, 53-59 (2016), hereby incorporated by reference in its entirety).

In order to eliminate the risk of PERV transmission from porcine organs to human hosts in pig-to-human xenotransplantation, intact PERVs must be inactivated and donor animals must be protected from reinfection by PERVs. Though it was considered difficult or impossible to eliminate PERV activity, an efficient method to inactivate all copies of PERVs in porcine cells using CRISPR-Cas9 has been previously reported (Yang, L. et al. Genome-wide inactivation of porcine endogenous retroviruses (PERVs). Science 350, 1101-1104 (2015), hereby incorporated by reference in its entirety). A PERV-inactive primary porcine cell could be used to clone pigs with genotypes identical to those of the original cell. However, these pigs would only remain PERV-inactive if protected from reinfection by PERVs.

There are three major subtypes of PERVs: PERV A, PERV B, and PERV C. Subtypes A and B are ubiquitous among pig strains and can be transmitted from porcine cells to other porcine cells and human cells. Subtype C is present only in some pig strains and can be transmitted among pigs (See, e.g., Denner, J., Specke, V., Thiesen, U., Karlas, A. & Kurth, R. Genetic alterations of the long terminal repeat of an ecotropic porcine endogenous retrovirus during passage in human cells. Virology 314, 125-133 (2003), hereby incorporated by reference in its entirety). In some cases, PERV C has been shown to recombine with PERV A and acquire critical residues of the PERV A envelope (env) gene that confer human-tropism (See, e.g., Patience, C. et al. Multiple Groups of Novel Retroviral Genomes in Pigs and Related Species. J. Virol. 75, 2771-2775 (2001), hereby incorporated by reference in its entirety). To date, only the porcine receptor for PERV-A, which mediates entry of PERV A and some PERV A/C recombinants, has been identified. The receptors for other PERV subtypes are still unknown. There still remains a need for methods of genetic modification that modulate/inactivate cellular receptors for PERV in PERV-free cells to protect these cells from reinfection by PERVs.

Genome editing and genetic modification of a cell via sequence-specific nucleases is known. A nuclease-mediated double-stranded DNA (dsDNA) break in the genome can be repaired by two main mechanisms: Non-Homologous End Joining (NHEJ), which frequently results in the introduction of non-specific insertions and deletions (indels), or homology directed repair (HDR), which incorporates a homologous strand as a repair template. When a sequence-specific nuclease is delivered along with a homologous donor DNA construct containing the desired mutations, gene targeting efficiencies are increased by 1000-fold compared to just the donor construct alone.

Alternative methods have been developed to accelerate the process of genome modification by directly injecting DNA or mRNA of site-specific nucleases into the one cell embryo to generate DNA double strand break (DSB) at a specified locus in various species. DSBs induced by these site-specific nucleases can then be repaired by either error-prone non-homologous end joining (NHEJ) resulting in mutant mice and rats carrying deletions or insertions at the cut site. If a donor plasmid with homology to the ends flanking the DSB is co-injected, high-fidelity homologous recombination can produce animals with targeted integrations. Because these methods require the complex designs of zinc finger nucleases (ZNFs) or Transcription activator-like effector nucleases (TALENs) for each target gene and because the efficiency of targeting may vary substantially, no multiplexed gene targeting has been reported to date.

Thus, there still remains a need for methods of producing genetically modified cells such as PERV-free cells with variant PERV receptor sequences to generate genetically modified tissues, organs and animals, such as pigs, for improved and safer sources of tissues and organs for transplantation.

### SUMMARY

Aspects of the present disclosure relate to genetic modification of a cell, including a porcine cell, such that the porcine receptor of PERV-A encoded by the SLC52A2 gene in the porcine cell is modified, for the purpose of selectively inhibiting entry of PERV-A and PERV-A/C in porcine cells and producing genetically modified porcine cell lines that are useful resources for xenotransplantation therapies. In some embodiments, the porcine cell is a PERV-inactive cell. In some embodiments, the disclosure provides the sequences of the genetic modifications used to render the PERV-A receptor non-permissive to PERV entry. In other embodiments, modified porcine cell lines, tissues, and animals that have the genetically modified alleles of SLC52A2 and one or more additional genetic modifications as suitable resources for xenotransplantation are provided.

Aspects of the present disclosure are directed the use of the Clustered Regularly Interspaced Short Palindromic Repeats (CRISPR) and CRISPR associated (Cas) proteins (CRISPR/Cas) system to achieve highly efficient and simultaneous targeting of multiple nucleic acid sequences in cells.

Aspects of the present disclosure are directed to the modification of genomic DNA, such as multiplex modification of DNA, in a cell (e.g., stem cell, somatic cell, germ line cell, zygote) using one or more guide RNAs (ribonucleic acids) to direct an enzyme having nuclease activity expressed by the cell, such as a DNA binding protein having nuclease activity, to a target location on the DNA (deoxyribonucleic acid) wherein the enzyme cuts the DNA and an exogenous donor nucleic acid is inserted into the DNA, such as by homologous recombination. Aspects of the present disclosure include cycling or repeating steps of DNA modification in a cell to create a cell having multiple modifications of DNA within the cell. Modifications can include insertion of exogenous donor nucleic acids. Modifications can include mutation and deletion of endogenous nucleic acids.

Multiple nucleic acid sequences can be modulated (e.g., inactivated) by a single step of introducing into a cell, which expresses an enzyme, and nucleic acids encoding a plurality of RNAs, such as by co-transformation, wherein the RNAs are expressed and wherein each RNA in the plurality guides the enzyme to a particular site of the DNA, the enzyme cuts the DNA. According to this aspect, many alterations or modification of the DNA in the cell are created in a single cycle.

According to one aspect, the cell expressing the enzyme has been genetically altered to express the enzyme such as by introducing into the cell a nucleic acid encoding the enzyme and which can be expressed by the cell. In this manner, aspects of the present disclosure include cycling the steps of introducing RNA into a cell which expresses the enzyme, introducing exogenous donor nucleic acid into the cell, expressing the RNA, forming a co-localization complex of the RNA, the enzyme and the DNA, and enzymatic cutting of the DNA by the enzyme. Insertion of a donor nucleic acid into the DNA is also provided herein. Cycling or repeating of the above steps results in multiplexed genetic modification of a cell at multiple loci, i.e., a cell having multiple genetic modifications.

According to certain aspects, DNA binding proteins or enzymes within the scope of the present disclosure include a protein that forms a complex with the guide RNA and with the guide RNA guiding the complex to a double stranded DNA sequence wherein the complex binds to the DNA sequence. According to one aspect, the enzyme can be an RNA guided DNA binding protein, such as an RNA guided DNA binding protein of a Type II CRISPR System that binds to the DNA and is guided by RNA. According to one aspect, the RNA guided DNA binding protein is a Cas9 protein.

This aspect of the present disclosure may be referred to as co-localization of the RNA and DNA binding protein to or with the double stranded DNA. In this manner, a DNA binding protein-guide RNA complex may be used to cut multiple sites of the double stranded DNA so as to create a cell with multiple genetic modifications, such as disruption of one or more (e.g., all) copies of a gene.

According to certain aspects, a method of making multiple alterations to target DNA in a cell expressing an enzyme that forms a co-localization complex with RNA complementary to the target DNA and that cleaves the target DNA in a site specific manner is provided including (a) introducing into the cell a first foreign nucleic acid encoding one or more RNAs complementary to the target DNA and which guide the enzyme to the target DNA, wherein the one or more RNAs and the enzyme are members of a co-localization complex for the target DNA, wherein the one or more RNAs and the enzyme co-localize to the target DNA, the enzyme cleaves the target DNA to produce altered DNA in the cell, and repeating step (a) multiple times to produce multiple alterations to the DNA in the cell.

In one aspect, a method of inactivating expression of one or more target nucleic acid sequences in a cell comprises introducing into a cell one or more ribonucleic acid (RNA) sequences that comprise a portion that is complementary to all or a portion of each of the one or more target nucleic acid sequences, and a nucleic acid sequence that encodes a Cas protein; and maintaining the cells under conditions in which the Cas protein is expressed and the Cas protein binds and inactivates the one or more target nucleic acid sequences in the cell.

In another aspect, a method of modifying a PERV-A receptor gene in a cell is provided. The method includes introducing into the cell a nucleic acid sequence encoding a Cas9 protein and a nucleic acid sequence encoding a guide RNA, and introducing into the cell a donor nucleic acid sequence, wherein the Cas9 protein and the guide RNA are expressed and co-localize at a genomic site near or in the PERV-A receptor gene and the donor nucleic acid sequence replaces the PERV-A receptor gene by homology directed repair (HDR).

In one aspect, a method of modulating one or more target nucleic acid sequences in a cell comprises introducing into the cell a nucleic acid sequence encoding an RNA complementary to all or a portion of a target nucleic acid sequence in the cell; introducing into the cell a nucleic acid sequence encoding an enzyme that interacts with the RNA and cleaves the target nucleic acid sequence in a site specific manner; and maintaining the cell under conditions in which the RNA binds to complementary target nucleic acid sequence forming a complex, and wherein the enzyme binds to a binding site on the complex and modulates the one or more target nucleic acid sequences.

In another aspect, a method of modifying expression of a PERV-A receptor gene in a cell. The method includes introducing into the cell a nucleic acid sequence encoding a fusion protein comprising a nuclease null Cas9 protein (dCas9) fused with a transcriptional repressor and a nucleic acid sequence encoding a guide RNA, wherein the fusion protein and the guide RNA are expressed and co-localize at a genomic site near or in the PERV-A receptor gene and modify the expression of the PERV-A receptor gene.

In the methods described herein, the introducing step can comprise transfecting the cell with the one or more RNA sequences and the nucleic acid sequence that encodes the Cas protein.

In the methods described herein, the introducing step can comprise transfecting the cell nucleic acid sequences that encode the one or more RNA sequences and the nucleic acid sequence that encodes the Cas protein.

In some embodiments, the one or more RNA sequences, the nucleic acid sequence that encodes the Cas protein, or a combination thereof are introduced into a genome of the cell. In some embodiments, the expression of the Cas protein is induced.

In the methods described, herein the cell is from an embryo. The cell can be a stem cell, zygote, or a germ line cell. In embodiments where the cell is a stem cell, the stem cell is an embryonic stem cell or pluripotent stem cell. In other embodiments, the cell is a somatic cell. In embodiments, where the cell is a somatic cell, the somatic cell is a eukaryotic cell or prokaryotic cell. The eukaryotic cell can be an animal cell, such as from a pig, mouse, rat, rabbit, dog, horse, cow, non-human primate, human. In some embodiments, the animal cell is a porcine cell. In other embodiments, the porcine cell is a porcine endogenous retrovirus (PERV)-inactive porcine fetal fibroblast cell (FF) or a PERV-inactive immortalized porcine kidney epithelial cell (PK). In the methods described herein, the one or more target nucleic acid sequences comprises a PERV-A receptor gene. In some embodiments, the one or more target nucleic acid sequences further comprises a second gene, GGTA1. In some embodiments, the PERV-A receptor gene comprises a SLC52A2 gene. In other embodiments, the SLC52A2 gene is inactivated. In exemplary embodiments, the SLC52A2 gene is inactivated by homology directed repair (HDR) wherein the SLC52A2 gene is replaced with a mutant SLC52A2 gene. In certain embodiments, the mutant SLC52A2 gene comprises a substitution of the Valine residue at position 109, including V109S, V109T, V109A, and V109P.

According to yet another aspect, the disclosure provides a method comprising modifying the SLC52A2 gene in a porcine cell to reduce or eliminate PERV-A binding. In certain embodiments, the modifying results in a V109S, V109T, V109A or V109P substitution. In other embodiments, the method further comprises modifying the GGTA1 gene.

In the methods described herein, the Cas protein is a Cas9. In some embodiments, the Cas9 is a Cas9 nickase or a nuclease null Cas9 (dCas9). In other embodiments, the dCas9 is further fused with a transcription repressor KRAB. In some embodiments, expression of the SLC52A2 gene is repressed by dCas9-KRAB. In other embodiments, the transcription or translation of the SLC52A2 gene is modified, diminished, or inhibited.

In some embodiments, the one or more RNA sequences can be about 10 to about 1000 nucleotides. For example, the one or more RNA sequences can be about 15 to about 200 nucleotides.

In some aspects, an engineered cell comprises one or more exogenous nucleic acid sequences that comprise a portion that is complementary to all or a portion of one or more target nucleic acid sequences of the cell; and a nucleic acid sequence that encodes a Cas protein, wherein the Cas protein is expressed and the Cas protein binds and inactivates the one or more target nucleic acid sequences of the cell.

In another aspect, an engineered cell comprises one or more exogenous nucleic acid sequences that comprise a portion that is complementary to all or a portion of one or more target nucleic acid sequences of the cell; and a nucleic acid sequence that encodes a Cas protein, wherein the Cas protein is expressed and the Cas protein binds and modulates the one or more target nucleic acid sequences of the cell.

In further aspect, the present disclosure provides tissues, organs or animals produced from the engineered cell according to the embodiments of the disclosure.

In another aspect, a nucleic acid sequence that comprises a portion that is complementary to all or a portion of one or more target nucleic acid sequences of the cell. In certain embodiments, the one or more target nucleic acid sequences comprise a porcine endogenous retrovirus (PERV) receptor A gene. In one embodiment, the PERV receptor A gene comprises a mutant SLC52A2 gene. In some embodiments, the mutant SLC52A2 gene comprises a substitution of the Valine residue at position 109, including V109S, V109T, V109A, and V109P.

In some embodiments, the engineered cell is a porcine cell. In other embodiments, the porcine cell is a primary cell. In other embodiments, the porcine cell is a porcine endogenous retrovirus (PERV)-inactive porcine fetal fibroblast cell (FF) or a PERV-inactive immortalized porcine kidney epithelial cell (PK).

In some embodiments, the one or more target nucleic acid sequences comprises a PERV-A receptor gene. In some embodiments, the one or more target nucleic acid sequences further comprises a second gene, such as GGTA1. In some embodiments, the PERV-A receptor gene comprises a SLC52A2 gene. In other embodiments, the SLC52A2 gene is inactivated. In exemplary embodiments, the SLC52A2 gene is inactivated by homology directed repair (HDR) wherein the SLC52A2 gene is replaced with a mutant SLC52A2 gene. In certain embodiments, the mutant SLC52A2 gene comprises a substitution of the Valine residue at position 109, including V109S, V109T, V109A, and V109P.

In some embodiments, the Cas protein is a Cas9. In some embodiments, the Cas9 is a Cas9 nickase or a nuclease null Cas9 (dCas9). In other embodiments, the dCas9 is further fused with a transcription repressor KRAB. In some embodiments, expression of the SLC52A2 gene is repressed by dCas9-KRAB. In other embodiments, the transcription or translation of the SLC52A2 gene is modified, diminished, or inhibited.

According to one aspect, the RNA is between about 10 to about 1000 nucleotides. According to one aspect, the RNA is between about 20 to about 100 nucleotides.

According to one aspect, the one or more RNAs is a guide RNA. According to one aspect, the one or more RNAs is a tracrRNA-crRNA fusion.

According to one aspect, the DNA is genomic DNA, mitochondrial DNA, viral DNA, or exogenous DNA.

Further features and advantages of certain embodiments of the present invention will become more fully apparent in the following description of embodiments and drawings thereof, and from the claims.

### DETAILED DESCRIPTION

Xenotransplantation is a promising strategy to solve the problem of the severe shortage of organs for human transplantation. However, the risk of cross-species transmission of porcine endogenous viruses (PERVs) has impeded the clinical application of xenotransplantation. The present disclosure provides methods and strategies of genetically modifying porcine cells that reduce the risk that porcine cells may be reinfected by PERVs and ultimately diminish the risk that PERVs may be transmitted from porcine cells, tissues, and organs to human cells. The production of PERV-inactive porcine cell lines using CRISPR-Cas9 genome engineering has been previously reported. The term “PERV-free” or “PERV-inactive” as used herein refers to cell lines in which all PERV genes are inactive, though they are still present in the genome. These PERV-free cells exhibited only background levels of PERV transmission to human cells. According to certain aspects, the present invention describes the production of primary porcine cells modified at the SLC52A2 locus, which encodes the porcine PERV-A receptor. This genetic modification renders the porcine cells nonpermissive to entry by PERV-A and the recombinant PERV-A/C. The disclosed methods represent an innovation towards the goal of delivering safe xeno-organs for human transplantation.

Aspects of the present invention are directed to the use of CRISPR/Cas9, for nucleic acid engineering. Described herein is the development of an efficient technology for the generation of animals (e.g., pigs) carrying multiple mutated genes. Specifically, the clustered regularly interspaced short palindromic repeats (CRISPR) and CRISPR associated genes (Cas genes), referred to herein as the CRISPR/Cas system, has been adapted as an efficient gene targeting technology e.g., for multiplexed genome editing. Demonstrated herein is that CRISPR/Cas mediated gene editing allows the simultaneous inactivation of the SLC52A2 gene, which encodes the porcine PERV-A receptor, and an additional locus, in PERV free cell such as a PERV free porcine fetal fibroblast cell line and in PERV-free immortalized porcine kidney epithelial cell line (e.g., PK15) with high efficiency. Co-injection or transfection of Cas9 mRNA and guide RNA (gRNA) targeting PERV receptor A into cells generated a PERV non-permissive cell line with biallelic mutations in both genes with an efficiency of up to 100%. Shown herein is that the CRISPR/Cas system allows the one step generation of cells carrying inactivation of multiple copies of target PERV genes. In certain embodiments a method described herein generates cell and animals, e.g., pigs, with inactivation of 1, 2, 3, 4, 5, or more genes with an efficiency of between 20% and 100%, e.g., at least 20%, 30%, 40%, 50%, 60%, 70%, 80%, 85%, 90%, 95%, or more, e.g., up to 96%, 97%, 98%, 99%, or more.

### Porcine PERV-A Receptor

The gene SLC52A2 (solute carrier family 52 member 2, NCBI Gene ID: 445519) encodes a protein that simultaneously functions as a riboflavin transporter and a receptor for PERV-A receptor (See, e.g., Denner, J. & Tönjes, R. R. Infection Barriers to Successful Xenotransplantation Focusing on Porcine Endogenous Retroviruses. Clin Microbiol Rev 25, 318-343 (2012), hereby incorporated by reference in its entirety). Since biallelic mutation of SLC52A2 causes Riboflavin Transporter Deficiency Neuronopathy in humans, with phenotypes including breathing problems due to paralysis of the diaphragm, weakness of the limbs, and cranial neuronopathy, inactivation of porcine SLC52A2 to prevent re-infection by PERV-A could potentially have negative effects on the health of the cells and organs of a pig (See, e.g., Denner, J. Recombinant porcine endogenous retroviruses (PERV-A/C): a new risk for xenotransplantation? Xenotransplantation 17, 120-120 (2010), hereby incorporated by reference in its entirety). The present disclosure devises a novel strategy to precisely modify SLC52A2 in such a way that the primary function as a riboflavin transporter is unaffected while the secondary function as an entry receptor for PERV A and PERV A/C is eliminated.

Unlike human and African green monkey cells, rhesus macaque, cynomolgus macaque, and baboon cells are not susceptible to PERV-A infection (See, e.g., Mattiuzzo, G. & Takeuchi, Y. Suboptimal Porcine Endogenous Retrovirus Infection in Non-Human Primate Cells: Implication for Preclinical Xenotransplantation. PLOS ONE 5, e13203 (2010), hereby incorporated by reference in its entirety, and FIGS. 1A-1B). It is believed that this is due to sequence differences at key regions of the genes encoding SLC52A2. The SLC52A2 homologs in rhesus macaques, cynomolgus macaques, and baboons encode serine at amino acid 109 in place of the leucine in human SLC52A2 (See, e.g., Mattiuzzo, G. & Takeuchi, Y. Suboptimal Porcine Endogenous Retrovirus Infection in Non-Human Primate Cells: Implication for Preclinical Xenotransplantation. PLOS ONE 5, e13203 (2010), hereby incorporated by reference in its entirety, and FIGS. 1A-1B). It has been demonstrated that substitution of L109S in the human PERV A receptor, HuPAR-1, results in elimination of PERV-A infection in vitro, whereas substituting S109 to L in the rhesus macaque PERV-A receptor, rhPAR-1, restores PERV-A infection (Mattiuzzo, G. & Takeuchi, Y. Suboptimal Porcine Endogenous Retrovirus Infection in Non-Human Primate Cells: Implication for Preclinical Xenotransplantation. PLOS ONE 5, e13203 (2010), hereby incorporated by reference in its entirety).

### CAS9 Description

RNA guided DNA binding proteins are readily known to those of skill in the art to bind to DNA for various purposes. Such DNA binding proteins may be naturally occurring. DNA binding proteins having nuclease activity are known to those of skill in the art, and include naturally occurring DNA binding proteins having nuclease activity, such as Cas9 proteins present, for example, in Type II CRISPR systems. Such Cas9 proteins and Type II CRISPR systems are well documented in the art. See Makarova et al., Nature Reviews, Microbiology, Vol. 9, June 2011, pp. 467-477 including all supplementary information hereby incorporated by reference in its entirety.

In general, bacterial and archaeal CRISPR-Cas systems rely on short guide RNAs in complex with Cas proteins to direct degradation of complementary sequences present within invading foreign nucleic acid. See Deltcheva, E. et al. CRISPR RNA maturation by trans-encoded small RNA and host factor RNase III. Nature 471, 602-607 (2011); Gasiunas, G., Barrangou, R., Horvath, P. & Siksnys, V. Cas9-crRNA ribonucleoprotein complex mediates specific DNA cleavage for adaptive immunity in bacteria. Proceedings of the National Academy of Sciences of the United States of America 109, E2579-2586 (2012); Jinek, M. et al. A programmable dual-RNA-guided DNA endonuclease in adaptive bacterial immunity. Science 337, 816-821 (2012); Sapranauskas, R. et al. The Streptococcus thermophilus CRISPR/Cas system provides immunity in Escherichia coli. Nucleic acids research 39, 9275-9282 (2011); and Bhaya, D., Davison, M. & Barrangou, R. CRISPR-Cas systems in bacteria and archaea: versatile small RNAs for adaptive defense and regulation. Annual review of genetics 45, 273-297 (2011). A recent in vitro reconstitution of the S. pyogenes type II CRISPR system demonstrated that crRNA (“CRISPR RNA”) fused to a normally trans-encoded tracrRNA (“trans-activating CRISPR RNA”) is sufficient to direct Cas9 protein to sequence-specifically cleave target DNA sequences matching the crRNA. Expressing a gRNA homologous to a target site results in Cas9 recruitment and degradation of the target DNA. See H. Deveau et al., Phage response to CRISPR-encoded resistance in Streptococcus thermophilus. Journal of Bacteriology 190, 1390 (February 2008).

Three classes of CRISPR systems are generally known and are referred to as Type I, Type II or Type III). According to one aspect, a particular useful enzyme according to the present disclosure to cleave dsDNA is the single effector enzyme, Cas9, common to Type II. See K. S. Makarova et al., Evolution and classification of the CRISPR-Cas systems. Nature reviews. Microbiology 9, 467 (June 2011) hereby incorporated by reference in its entirety. Within bacteria, the Type II effector system consists of a long pre-crRNA transcribed from the spacer-containing CRISPR locus, the multifunctional Cas9 protein, and a tracrRNA important for gRNA processing. The tracrRNAs hybridize to the repeat regions separating the spacers of the pre-crRNA, initiating dsRNA cleavage by endogenous RNase III, which is followed by a second cleavage event within each spacer by Cas9, producing mature crRNAs that remain associated with the tracrRNA and Cas9. TracrRNA-crRNA fusions are contemplated for use in the present methods.

According to one aspect, the enzyme of the present disclosure, such as Cas9 unwinds the DNA duplex and searches for sequences matching the crRNA to cleave. Target recognition occurs upon detection of complementarity between a “protospacer” sequence in the target DNA and the remaining spacer sequence in the crRNA. Importantly, Cas9 cuts the DNA only if a correct protospacer-adjacent motif (PAM) is also present at the 3′ end. According to certain aspects, different protospacer-adjacent motif can be utilized. For example, the S. pyogenes system requires an NGG sequence, where N can be any nucleotide. S. thermophilus Type II systems require NGGNG (see P. Horvath, R. Barrangou, CRISPR/Cas, the immune system of bacteria and archaea. Science 327, 167 (Jan. 8, 2010) hereby incorporated by reference in its entirety and NNAGAAW (see H. Deveau et al., Phage response to CRISPR-encoded resistance in Streptococcus thermophilus. Journal of bacteriology 190, 1390 (February 2008) hereby incorporated by reference in its entirety), respectively, while different S. mutans systems tolerate NGG or NAAR (see J. R. van der Ploeg, Analysis of CRISPR in Streptococcus mutans suggests frequent occurrence of acquired immunity against infection by M102-like bacteriophages. Microbiology 155, 1966 (June 2009) hereby incorporated by reference in its entirety. Bioinformatic analyses have generated extensive databases of CRISPR loci in a variety of bacteria that may serve to identify additional useful PAMs and expand the set of CRISPR-targetable sequences (see M. Rho, Y. W. Wu, H. Tang, T. G. Doak, Y. Ye, Diverse CRISPRs evolving in human microbiomes. PLoS genetics 8, e1002441 (2012) and D. T. Pride et al., Analysis of streptococcal CRISPRs from human saliva reveals substantial sequence diversity within and between subjects over time. Genome research 21, 126 (January 2011) each of which are hereby incorporated by reference in their entireties.

In S. pyogenes, Cas9 generates a blunt-ended double-stranded break 3 bp upstream of the protospacer-adjacent motif (PAM) via a process mediated by two catalytic domains in the protein: an HNH domain that cleaves the complementary strand of the DNA and a RuvC-like domain that cleaves the non-complementary strand. See Jinek et al., Science 337, 816-821 (2012) hereby incorporated by reference in its entirety. Cas9 proteins are known to exist in many Type II CRISPR systems including the following as identified in the supplementary information to Makarova et al., Nature Reviews, Microbiology, Vol. 9, June 2011, pp. 467-477: Methanococcus maripaludis C7; Corynebacterium diphtheriae; Corynebacterium efficiens YS-314; Corynebacterium glutamicum ATCC 13032 Kitasato; Corynebacterium glutamicum ATCC 13032 Bielefeld; Corynebacterium glutamicum R; Corynebacterium kroppenstedtii DSM 44385; Mycobacterium abscessus ATCC 19977; Nocardia farcinica IFM10152; Rhodococcus erythropolis PR4; Rhodococcus jostii RHA1; Rhodococcus opacus B4 uid36573; Acidothermus cellulolyticus 11B; Arthrobacter chlorophenolicus A6; Kribbella flavida DSM 17836 uid43465; Thermomonospora curvata DSM 43183; Bifidobacterium dentium Bd1; Bifidobacterium longum DJO10A; Slackia heliotrinireducens DSM 20476; Persephonella marina EX H1; Bacteroides fragilis NCTC 9434; Capnocytophaga ochracea DSM 7271; Flavobacterium psychrophilum JIP02 86; Akkermansia muciniphila ATCC BAA 835; Roseiflexus castenholzii DSM 13941; Roseiflexus RS1; Synechocystis PCC6803; Elusimicrobium minutum Pei191; uncultured Termite group 1 bacterium phylotype Rs D17; Fibrobacter succinogenes S85; Bacillus cereus ATCC 10987; Listeria innocua; Lactobacillus casei; Lactobacillus rhamnosus GG; Lactobacillus salivarius UCC118; Streptococcus agalactiae A909; Streptococcus agalactiae NEM316; Streptococcus agalactiae 2603; Streptococcus dysgalactiae equisimilis GGS 124; Streptococcus equi zooepidemicus MGCS10565; Streptococcus gallolyticus UCN34 uid46061; Streptococcus gordonii Challis subst CH1; Streptococcus mutans NN2025 uid46353; Streptococcus mutans; Streptococcus pyogenes M1 GAS; Streptococcus pyogenes MGAS5005; Streptococcus pyogenes MGAS2096; Streptococcus pyogenes MGAS9429; Streptococcus pyogenes MGAS10270; Streptococcus pyogenes MGAS6180; Streptococcus pyogenes MGAS315; Streptococcus pyogenes SSI-1; Streptococcus pyogenes MGAS10750; Streptococcus pyogenes NZ131; Streptococcus thermophiles CNRZ1066; Streptococcus thermophiles LMD-9; Streptococcus thermophiles LMG 18311; Clostridium botulinum A3 Loch Maree; Clostridium botulinum B Eklund 17B; Clostridium botulinum Ba4 657; Clostridium botulinum F Langeland; Clostridium cellulolyticum H10; Finegoldia magna ATCC 29328; Eubacterium rectale ATCC 33656; Mycoplasma gallisepticum; Mycoplasma mobile 163K; Mycoplasma penetrans; Mycoplasma synoviae 53; Streptobacillus moniliformis DSM 12112; Bradyrhizobium BTAi1; Nitrobacter hamburgensis X14; Rhodopseudomonas palustris BisB18; Rhodopseudomonas palustris BisB5; Parvibaculum lavamentivorans DS-1; Dinoroseobacter shibae DFL 12; Gluconacetobacter diazotrophicus Pal 5 FAPERJ; Gluconacetobacter diazotrophicus Pal 5 JGI; Azospirillum B510 uid46085; Rhodospirillum rubrum ATCC 11170; Diaphorobacter TPSY uid29975; Verminephrobacter eiseniae EF01-2; Neisseria meningitides 053442; Neisseria meningitides alpha14; Neisseria meningitides Z2491; Desulfovibrio salexigens DSM 2638; Campylobacter jejuni doylei 269 97; Campylobacter jejuni 81116; Campylobacter jejuni; Campylobacter lari RM2100; Helicobacter hepaticus; Wolinella succinogenes; Tolumonas auensis DSM 9187; Pseudoalteromonas atlantica T6c; Shewanella pealeana ATCC 700345; Legionella pneumophila Paris; Actinobacillus succinogenes 130Z; Pasteurella multocida; Francisella tularensis novicida U112; Francisella tularensis holarctica; Francisella tularensis FSC 198; Francisella tularensis tularensis; Francisella tularensis WY96-3418; and Treponema denticola ATCC 35405. The Cas9 protein may be referred by one of skill in the art in the literature as Csn1. An exemplary S. pyogenes Cas9 protein sequence is provided in Deltcheva et al., Nature 471, 602-607 (2011) hereby incorporated by reference in its entirety.

Modification to the Cas9 protein is contemplated by the present disclosure. CRISPR systems useful in the present disclosure are described in R. Barrangou, P. Horvath, CRISPR: new horizons in phage resistance and strain identification. Annual review of food science and technology 3, 143 (2012) and B. Wiedenheft, S. H. Sternberg, J. A. Doudna, RNA-guided genetic silencing systems in bacteria and archaea. Nature 482, 331 (Feb. 16, 2012) each of which are hereby incorporated by reference in their entireties.

According to certain aspects, the DNA binding protein is altered or otherwise modified to inactivate the nuclease activity. Such alteration or modification includes altering one or more amino acids to inactivate the nuclease activity or the nuclease domain. Such modification includes removing the polypeptide sequence or polypeptide sequences exhibiting nuclease activity, i.e. the nuclease domain, such that the polypeptide sequence or polypeptide sequences exhibiting nuclease activity, i.e. nuclease domain, are absent from the DNA binding protein. Other modifications to inactivate nuclease activity will be readily apparent to one of skill in the art based on the present disclosure. Accordingly, a nuclease-null DNA binding protein includes polypeptide sequences modified to inactivate nuclease activity or removal of a polypeptide sequence or sequences to inactivate nuclease activity. The nuclease-null DNA binding protein retains the ability to bind to DNA even though the nuclease activity has been inactivated. Accordingly, the DNA binding protein includes the polypeptide sequence or sequences required for DNA binding but may lack the one or more or all of the nuclease sequences exhibiting nuclease activity. Accordingly, the DNA binding protein includes the polypeptide sequence or sequences required for DNA binding but may have one or more or all of the nuclease sequences exhibiting nuclease activity inactivated.

According to one aspect, a DNA binding protein having two or more nuclease domains may be modified or altered to inactivate all but one of the nuclease domains. Such a modified or altered DNA binding protein is referred to as a DNA binding protein nickase, to the extent that the DNA binding protein cuts or nicks only one strand of double stranded DNA. When guided by RNA to DNA, the DNA binding protein nickase is referred to as an RNA guided DNA binding protein nickase. An exemplary DNA binding protein is an RNA guided DNA binding protein nuclease of a Type II CRISPR System, such as a Cas9 protein or modified Cas9 or homolog of Cas9. An exemplary DNA binding protein is a Cas9 protein nickase. An exemplary DNA binding protein is an RNA guided DNA binding protein of a Type II CRISPR System which lacks nuclease activity. An exemplary DNA binding protein is a nuclease-null or nuclease deficient Cas9 protein.

According to an additional aspect, nuclease-null Cas9 proteins are provided where one or more amino acids in Cas9 are altered or otherwise removed to provide nuclease-null Cas9 proteins. According to one aspect, the amino acids include D10 and H840. See Jinek et al., Science 337, 816-821 (2012). According to an additional aspect, the amino acids include D839 and N863. According to one aspect, one or more or all of D10, H840, D839 and H863 are substituted with an amino acid which reduces, substantially eliminates or eliminates nuclease activity. According to one aspect, one or more or all of D10, H840, D839 and H863 are substituted with alanine. According to one aspect, a Cas9 protein having one or more or all of D10, H840, D839 and H863 substituted with an amino acid which reduces, substantially eliminates or eliminates nuclease activity, such as alanine, is referred to as a nuclease-null Cas9 (“Cas9Nuc”) and exhibits reduced or eliminated nuclease activity, or nuclease activity is absent or substantially absent within levels of detection. According to this aspect, nuclease activity for a Cas9Nuc may be undetectable using known assays, i.e. below the level of detection of known assays.

According to one aspect, the Cas9 protein, Cas9 protein nickase or nuclease null Cas9 includes homologs and orthologs thereof which retain the ability of the protein to bind to the DNA and be guided by the RNA. According to one aspect, the Cas9 protein includes the sequence as set forth for naturally occurring Cas9 from S. thermophiles or S. pyogenes and protein sequences having at least 30%, 40%, 50%, 60%, 70%, 80%, 90%, 95%, 98% or 99% homology thereto and being a DNA binding protein, such as an RNA guided DNA binding protein.

An exemplary CRISPR system includes the S. thermophiles Cas9 nuclease (ST1 Cas9) (see Esvelt K M, et al., Orthogonal Cas9 proteins for RNA-guided gene regulation and editing, Nature Methods., (2013) hereby incorporated by reference in its entirety). An exemplary CRISPR system includes the S. pyogenes Cas9 nuclease (Sp. Cas9), an extremely high-affinity (see Sternberg, S. H., Redding, S., Jinek, M., Greene, E. C. & Doudna, J. A. DNA interrogation by the CRISPR RNA-guided endonuclease Cas9. Nature 507, 62-67 (2014) hereby incorporated by reference in its entirety), programmable DNA-binding protein isolated from a type II CRISPR-associated system (see Garneau, J. E. et al. The CRISPR/Cas bacterial immune system cleaves bacteriophage and plasmid DNA. Nature 468, 67-71 (2010) and Jinek, M. et al. A programmable dual-RNA-guided DNA endonuclease in adaptive bacterial immunity. Science 337, 816-821 (2012) each of which are hereby incorporated by reference in its entirety). According to certain aspects, a nuclease null or nuclease deficient Cas 9 can be used in the methods described herein. Such nuclease null or nuclease deficient Cas9 proteins are described in Gilbert, L. A. et al. CRISPR-mediated modular RNA-guided regulation of transcription in eukaryotes. Cell 154, 442-451 (2013); Mali, P. et al. CAS9 transcriptional activators for target specificity screening and paired nickases for cooperative genome engineering. Nature biotechnology 31, 833-838 (2013); Maeder, M. L. et al. CRISPR RNA-guided activation of endogenous human genes. Nature methods 10, 977-979 (2013); and Perez-Pinera, P. et al. RNA-guided gene activation by CRISPR-Cas9-based transcription factors. Nature methods 10, 973-976 (2013) each of which are hereby incorporated by reference in its entirety. The DNA locus targeted by Cas9 (and by its nuclease-deficient mutant, “dCas9” precedes a three nucleotide (nt) 5′-NGG-3′ “PAM” sequence, and matches a 15-22-nt guide or spacer sequence within a Cas9-bound RNA cofactor, referred to herein and in the art as a guide RNA. Altering this guide RNA is sufficient to target Cas9 or a nuclease deficient Cas9 to a target nucleic acid. In a multitude of CRISPR-based biotechnology applications (see Mali, P., Esvelt, K. M. & Church, G. M. Cas9 as a versatile tool for engineering biology. Nature methods 10, 957-963 (2013); Hsu, P. D., Lander, E. S. & Zhang, F. Development and Applications of CRISPR-Cas9 for Genome Engineering. Cell 157, 1262-1278 (2014); Chen, B. et al. Dynamic imaging of genomic loci in living human cells by an optimized CRISPR/Cas system. Cell 155, 1479-1491 (2013); Shalem, O. et al. Genome-scale CRISPR-Cas9 knockout screening in human cells. Science 343, 84-87 (2014); Wang, T., Wei, J. J., Sabatini, D. M. & Lander, E. S. Genetic screens in human cells using the CRISPR-Cas9 system. Science 343, 80-84 (2014); Nissim, L., Perli, S. D., Fridkin, A., Perez-Pinera, P. & Lu, T. K. Multiplexed and Programmable Regulation of Gene Networks with an Integrated RNA and CRISPR/Cas Toolkit in Human Cells. Molecular cell 54, 698-710 (2014); Ryan, O. W. et al. Selection of chromosomal DNA libraries using a multiplex CRISPR system. eLife 3 (2014); Gilbert, L. A. et al. Genome-Scale CRISPR-Mediated Control of Gene Repression and Activation. Cell (2014); and Citorik, R. J., Mimee, M. & Lu, T. K. Sequence-specific antimicrobials using efficiently delivered RNA-guided nucleases. Nature biotechnology (2014) each of which are hereby incorporated by reference in its entirety), the guide is often presented in a so-called sgRNA (single guide RNA), wherein the two natural Cas9 RNA cofactors (gRNA and tracrRNA) are fused via an engineered loop or linker.

According to one aspect, the Cas9 protein is an enzymatically active Cas9 protein, a Cas9 protein wild-type protein, a Cas9 protein nickase or a nuclease null or nuclease deficient Cas9 protein. Additional exemplary Cas9 proteins include Cas9 proteins attached to, bound to or fused with functional proteins such as transcriptional regulators, such as transcriptional activators or repressors, a Fok-domain, such as Fok 1, an aptamer, a binding protein, PP7, MS2 and the like.

According to certain aspects, the Cas9 protein may be delivered directly to a cell by methods known to those of skill in the art, including injection or lipofection, or as translated from its cognate mRNA, or transcribed from its cognate DNA into mRNA (and thereafter translated into protein). Cas9 DNA and mRNA may be themselves introduced into cells through electroporation, transient and stable transfection (including lipofection) and viral transduction or other methods known to those of skill in the art.

### Guide RNA Description

Embodiments of the present disclosure are directed to the use of a CRISPR/Cas system and, in particular, a guide RNA which may include one or more of a spacer sequence, a tracr mate sequence and a tracr sequence. The term spacer sequence is understood by those of skill in the art and may include any polynucleotide having sufficient complementarity with a target nucleic acid sequence to hybridize with the target nucleic acid sequence and direct sequence-specific binding of a CRISPR complex to the target sequence. The guide RNA may be formed from a spacer sequence covalently connected to a tracr mate sequence (which may be referred to as a crRNA) and a separate tracr sequence, wherein the tracr mate sequence is hybridized to a portion of the tracr sequence. According to certain aspects, the tracr mate sequence and the tracr sequence are connected or linked such as by covalent bonds by a linker sequence, which construct may be referred to as a fusion of the tracr mate sequence and the tracr sequence. The linker sequence referred to herein is a sequence of nucleotides, referred to herein as a nucleic acid sequence, which connect the tracr mate sequence and the tracr sequence. Accordingly, a guide RNA may be a two component species (i.e., separate crRNA and tracr RNA which hybridize together) or a unimolecular species (i.e., a crRNA-tracr RNA fusion, often termed an sgRNA).

According to certain aspects, the guide RNA is between about 10 to about 500 nucleotides. According to one aspect, the guide RNA is between about 20 to about 100 nucleotides. According to certain aspects, the spacer sequence is between about 10 and about 500 nucleotides in length. According to certain aspects, the tracr mate sequence is between about 10 and about 500 nucleotides in length. According to certain aspects, the tracr sequence is between about 10 and about 100 nucleotides in length. According to certain aspects, the linker nucleic acid sequence is between about 10 and about 100 nucleotides in length.

According to one aspect, embodiments described herein include guide RNA having a length including the sum of the lengths of a spacer sequence, tracr mate sequence, tracr sequence, and linker sequence (if present). Accordingly, such a guide RNA may be described by its total length which is a sum of its spacer sequence, tracr mate sequence, tracr sequence, and linker sequence (if present). According to this aspect, all of the ranges for the spacer sequence, tracr mate sequence, tracr sequence, and linker sequence (if present) are incorporated herein by reference and need not be repeated. A guide RNA as described herein may have a total length based on summing values provided by the ranges described herein. Aspects of the present disclosure are directed to methods of making such guide RNAs as described herein by expressing constructs encoding such guide RNA using promoters and terminators and optionally other genetic elements as described herein.

According to certain aspects, the guide RNA may be delivered directly to a cell as a native species by methods known to those of skill in the art, including injection or lipofection, or as transcribed from its cognate DNA, with the cognate DNA introduced into cells through electroporation, transient and stable transfection (including lipofection) and viral transduction.

### Donor Description

The term “donor nucleic acid” include a nucleic acid sequence which is to be inserted into genomic DNA according to methods described herein. The donor nucleic acid sequence may be expressed by the cell.

According to one aspect, the donor nucleic acid is exogenous to the cell. According to one aspect, the donor nucleic acid is foreign to the cell. According to one aspect, the donor nucleic acid is non-naturally occurring within the cell.

### Transcription Regulator Description

According to one aspect, an engineered Cas9-gRNA system is provided which enables RNA-guided DNA regulation in cells by tethering transcriptional activation/repression domains to either a nuclease-null Cas9 or to guide RNAs. According to one aspect of the present disclosure, one or more transcriptional regulatory proteins or domains (such terms are used interchangeably) are joined or otherwise connected to a nuclease-deficient Cas9 or one or more guide RNA (gRNA). The transcriptional regulatory domains correspond to targeted loci. Accordingly, aspects of the present disclosure include methods and materials for localizing transcriptional regulatory domains to targeted loci by fusing, connecting or joining such domains to either Cas9N or to the gRNA.

### Foreign Nucleic Acids Description

Foreign nucleic acids (i.e. those which are not part of a cell's natural nucleic acid composition) may be introduced into a cell using any method known to those skilled in the art for such introduction. Such methods include transfection, transduction, viral transduction, microinjection, lipofection, nucleofection, nanoparticle bombardment, transformation, conjugation and the like. One of skill in the art will readily understand and adapt such methods using readily identifiable literature sources.

### Cells

Cells according to the present disclosure include any cell into which foreign nucleic acids can be introduced and expressed as described herein. It is to be understood that the basic concepts of the present disclosure described herein are not limited by cell type. In some embodiments, the cell is from an embryo. The cell can be a stem cell, zygote, or a germ line cell. In embodiments where the cell is a stem cell, the stem cell is an embryonic stem cell or pluripotent stem cell. In other embodiments, the cell is a somatic cell. In embodiments, where the cell is a somatic cell, the somatic cell is a eukaryotic cell or prokaryotic cell. The eukaryotic cell can be an animal cell, such as from a pig, mouse, rat, rabbit, dog, horse, cow, non-human primate, human. In some embodiments, the animal cell is a porcine cell. In other embodiments, the porcine cell is a porcine endogenous retrovirus (PERV)-free porcine fetal fibroblast cell (FF) or a PERV-free immortalized porcine kidney epithelial cell (PK).

### Vectors

Vectors are contemplated for use with the methods and constructs described herein. The term “vector” includes a nucleic acid molecule capable of transporting another nucleic acid to which it has been linked. Vectors used to deliver the nucleic acids to cells as described herein include vectors known to those of skill in the art and used for such purposes. Certain exemplary vectors may be plasmids, lentiviruses or adeno-associated viruses known to those of skill in the art. Vectors include, but are not limited to, nucleic acid molecules that are single-stranded, doublestranded, or partially double-stranded; nucleic acid molecules that comprise one or more free ends, no free ends (e.g. circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g. retroviruses, lentiviruses, replication defective retroviruses, adenoviruses, replication defective adenoviruses, and adeno-associated viruses). Viral vectors also include polynucleotides carried by a virus for transfection into a host cell. Certain vectors are capable of autonomous replication in a host cell into which they are introduced (e.g. bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors.” Common expression vectors of utility in recombinant DNA techniques are often in the form of plasmids. Recombinant expression vectors can comprise a nucleic acid of the invention in a form suitable for expression of the nucleic acid in a host cell, which means that the recombinant expression vectors include one or more regulatory elements, which may be selected on the basis of the host cells to be used for expression, that is operatively-linked to the nucleic acid sequence to be expressed. Within a recombinant expression vector, “operably linked” is intended to mean that the nucleotide sequence of interest is linked to the regulatory element(s) in a manner that allows for expression of the nucleotide sequence (e.g. in an in vitro transcription/translation system or in a host cell when the vector is introduced into the host cell).

Methods of non-viral delivery of nucleic acids or native DNA binding protein, native guide RNA or other native species include lipofection, microinjection, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Felgner, WO 91/17424; WO 91/16024. Delivery can be to cells (e.g. in vitro or ex vivo administration) or target tissues (e.g. in vivo administration). The term native includes the protein, enzyme or guide RNA species itself and not the nucleic acid encoding the species.

### Regulatory Elements and Terminators and Tags

Regulatory elements are contemplated for use with the methods and constructs described herein. The term “regulatory element” is intended to include promoters, enhancers, internal ribosomal entry sites (IRES), and other expression control elements (e.g. transcription termination signals, such as polyadenylation signals and poly-U sequences). Such regulatory elements are described, for example, in Goeddel, GENE EXPRESSION TECHNOLOGY: METHODS IN ENZYMOLOGY 185, Academic Press, San Diego, Calif. (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter may direct expression primarily in a desired tissue of interest, such as muscle, neuron, bone, skin, blood, specific organs (e.g. liver, pancreas), or particular cell types (e.g. lymphocytes). Regulatory elements may also direct expression in a temporal-dependent manner, such as in a cell-cycle dependent or developmental stage-dependent manner, which may or may not also be tissue or cell-type specific. In some embodiments, a vector may comprise one or more pol III promoter (e.g. 1, 2, 3, 4, 5, or more pol III promoters), one or more pol II promoters (e.g. 1, 2, 3, 4, 5, or more pol II promoters), one or more pol I promoters (e.g. 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer) [see, e.g., Boshart et al, Cell, 41:521-530 (1985)], the SV40 promoter, the dihydrofolate reductase promoter, the β-actin promoter, the phosphoglycerol kinase (PGK) promoter, and the EF1α promoter and Pol II promoters described herein. Also encompassed by the term “regulatory element” are enhancer elements, such as WPRE; CMV enhancers; the R-U5′ segment in LTR of HTLV-I (Mol. Cell. Biol., Vol. 8(1), p. 466-472, 1988); SV40 enhancer; and the intron sequence between exons 2 and 3 of rabbit β-globin (Proc. Natl. Acad. Sci. USA., Vol. 78(3), p. 1527-31, 1981). It will be appreciated by those skilled in the art that the design of the expression vector can depend on such factors as the choice of the host cell to be transformed, the level of expression desired, etc. A vector can be introduced into host cells to thereby produce transcripts, proteins, or peptides, including fusion proteins or peptides, encoded by nucleic acids as described herein (e.g., clustered regularly interspersed short palindromic repeats (CRISPR) transcripts, proteins, enzymes, mutant forms thereof, fusion proteins thereof, etc.).

Aspects of the methods described herein may make use of terminator sequences. A terminator sequence includes a section of nucleic acid sequence that marks the end of a gene or operon in genomic DNA during transcription. This sequence mediates transcriptional termination by providing signals in the newly synthesized mRNA that trigger processes which release the mRNA from the transcriptional complex. These processes include the direct interaction of the mRNA secondary structure with the complex and/or the indirect activities of recruited termination factors. Release of the transcriptional complex frees RNA polymerase and related transcriptional machinery to begin transcription of new mRNAs. Terminator sequences include those known in the art and identified and described herein.

Aspects of the methods described herein may make use of epitope tags and reporter gene sequences. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporter genes include, but are not limited to, glutathione-S-transferase (GST), horseradish peroxidase (HRP), chloramphenicol acetyltransferase (CAT) beta-galactosidase, betaglucuronidase, luciferase, green fluorescent protein (GFP), HcRed, DsRed, cyan fluorescent protein (CFP), yellow fluorescent protein (YFP), and autofluorescent proteins including blue fluorescent protein (BFP).

The following examples are set forth as being representative of the present disclosure. These examples are not to be construed as limiting the scope of the present disclosure as these and other equivalent embodiments will be apparent in view of the present disclosure and accompanying claims.

### EXEMPLIFICATION

### Example I

### The Production of Primary and Immortalized Porcine Cells with Genetically Modified Alleles of SLC52A2

The porcine SLC52A2 gene encodes the porcine PERV-A receptor. It is anticipated that porcine cells having genetically modified SLC52A2 gene will render these cells resistant to reinfection by PERV-A and PERV-A/C subtypes, thereby diminishing the risk of transmitting PERVs to human cells and are useful resources in xenotransplantation applications. In 2015, a method for the genome-wide inactivation of PERVs in a porcine cell line has been demonstrated (See, e.g., Yang, L. et al. Genome-wide inactivation of porcine endogenous retroviruses (PERVs). Science 350, 1101-1104 (2015), hereby incorporated by reference in its entirety). In this example, a novel strategy to protect porcine cell line and pigs from re-infection by PERV-A and PERV-A/C is presented.

A precise biallelic mutation in SLC52A2 that produces an amino acid substitution of V109S is introduced in PERV-free porcine fetal fibroblast cells and in PERV-free immortalized porcine kidney epithelial cells. This mutation is designed to eliminate the function of SLC52A2 as a PERV receptor while keeping its function as a riboflavin transporter undisturbed. Additional amino acid substitution such as V109T, V109A or V109P can also be introduced since the amino acids proline and threonine have biochemical properties similar to those of serine, and since alanine is a relatively unreactive amino acid that may eliminate the activity of the critical region of the PERV-A receptor.

This genetic modification is made using CRISPR-Cas9 and homology-directed repair (HDR). A plasmid homology donor template DNA encoding a substitution mutation of GTG→AGT is transfected together with a plasmid encoding a nuclease such as Cas9 to cells to increase the efficiency of HDR. CRISPR-Cas9 and HDR will introduce targeted DNA damage in the genome. When the DNA damage is repaired by the HDR, the template DNA may be used to precisely modify the original sequence. A single stranded oligonucleotide can also be used as a template donor, though plasmid donors are known to increase the efficiency of HDR.

Since cells that undergo multiplex genetic modification and exhibit mutation at a given locus are more likely to have mutations at a second locus, a plasmid encoding additional guide RNAs targeting GGTA1 is transfected. GGTA1 is the porcine gene encoding α1,3-galactosyltransferase (GGTA1), which produces the cell-surface glycoprotein galactose-α1,3-galactose. A beads-based enrichment strategy is used to isolate GGTA1-null cells. The enriched populations of genetically modified PK15 and FF cells are single cell sorted, and individual colonies are genotyped in order to identify biallelic PERV-A receptor mutants. An HDR efficiency of 30% is achieved and clones with biallelic modifications of SLC52A2 in both the PERV-free PK15 and PERV-free FF cell populations is isolated. It is anticipated that, when co-cultured with PERV-free PK15 and PERV-free FF, these cells having biallelic PERV-A receptor mutants will be less susceptible to reinfection by PERVs.

In addition to amino acid substitution, porcine SLC52A2 gene can be inactivated or disrupted in other ways to confer to porcine cells resistance to PERV-A for applications of xenotransplantation. For example, the inactivation or disruption of porcine SLC52A2 can be achieved by disrupting transcription or translation of the gene. Three techniques to accomplish this are: (i) using genome editing technologies to modify the start codon so that transcription is not properly initiated, (ii) using genome editing technologies to disrupt the first exon of the gene with the intention of producing a frameshift mutation that disrupts proper translation of the gene, and (iii) specifically repressing transcription of SLC52A2 using effector proteins, e.g. dCas9-KRAB.

Further, the disruption of porcine SLC52A2 can also be achieved by disrupting the structure of the protein in order to interfere with its endogenous function. This can be accomplished by modifying the transmembrane domains of the receptor in order to render it non-permissive to PERV-A or by disrupting the cell surface localization signal in order to prevent the receptor from reaching the cell membrane. The human homolog of SLC52A2 is thought to contain ten or eleven putative transmembrane domains, and the corresponding transmembrane domains in porcine SLC52A2 can be targeted for mutation. (http://www.omim.org/entry/607882). Targeted disruption of SLC52A2 to modify the structure of the protein can also be accomplished by modifying the functional domain of the receptor.

The disruption of porcine SLC52A2 can be accomplished by various targeted genome engineering technologies. Several tools are available that can be used to achieve the genetic modifications described above. These include nucleases that introduce double stranded breaks in DNA, e.g. Zinc Finger Nucleases, TAL effector nucleases, CRISPR associated nucleases including CRISPR-Cas9 and CRISPR-Cpf1.

Other tools for targeted genetic modification include nickases, which introduce single stranded breaks in DNA, and deaminases, which modify certain nucleotides.

Table of constructs used:

Guide RNAs used to target GGTA:
GGTA3 gRNA:GAGAAAATAATGAATGTCAA
Sequence altered in porcine SLC52A2, with altered nucleotides in bold:
CGCAGCAGGCCAGCGCCAACACAAAGGTCAGGGCCAGGAAGGCCACAGAGTGACT
CTGTCCTGCCATCACTGTCAGGTGTTGCCACAGTG
to
CGCAGCAGGCCAGCGCCAACACAAAGGTCAGGGCCAGGAAGGCCACAGAAGTACT
CTGTCCTGCCATCACTGTCAGGTGTTGCCACAGTG

The genetically modified cell lines produced by the methods disclosed herein are useful foundational cell lines for further modification for applications of xenotransplantation. For example, pigs cloned from these engineered cell lines is anticipated to exhibit resistance to infection by PERV-A, one of the major subtypes of PERVs, while otherwise maintaining good health. Healthy organs transplanted from these pigs to humans will be immune to future infection by PERV-A that could reinstate the risk of zoonosis from the transplanted organ to the human host.

Methods and strategies disclosed in the present disclosure represent a generalizable strategy for genetic modification for the purpose of diminishing or preventing infection by retroviruses or pathogens whose entry is mediated by cell surface receptors. For example, the methods for genetically modifying SLC52A2 for the purpose of diminishing the risk of reinfection by PERV-A and PERV-A/C may be broadly applied in many applications to reduce or prevent cellular entry by pathogens by genetically modifying the cell-surface receptors that mediate such entry.

### Example II

### Methods

### Cell Culture

PK15 cells are maintained in Dulbecco's modified Eagle's medium (DMEM, Invitrogen) high glucose with sodium pyruvate supplemented with 10% fetal bovine serum (Invitrogen), and 1% penicillin/streptomycin (Pen/Strep, Invitrogen). FF cells are maintained in Dulbecco's modified Eagle's medium (DMEM, Invitrogen) high glucose with sodium pyruvate supplemented with 15% fetal bovine serum (Invitrogen), 1% HEPES, and 1% penicillin/streptomycin (Pen/Strep, Invitrogen). All cells are maintained in a humidified incubator at 37° C. and 5% CO₂. PFFF3 were maintained in Dulbecco's modified Eagle's medium (DMEM, Invitrogen) high glucose with sodium pyruvate supplemented with 15% fetal bovine serum (Invitrogen), and 1% penicillin/streptomycin (Pen/Strep, Invitrogen). All cells were maintained in a humidified incubator at 37° C. and 5% CO₂.

### Transfection

PERV A receptor gRNA (5′-CCTGACAGTGATGGCAGGAC-3′), PERV A receptor HDR donor, GGTA3 gRNA (5′-GAGAAAATAATGAATGTCAA-3′), and LentiCRISPR V2 (Addene #52961) were combined in equimolar ratios and delivered to cells via Lipofectamine 2000 (Invitrogen) transfection according to the manufacturer's instructions. Briefly, DNA was resuspended in 500 μL Opti-MEM (Invitrogen), Lipofectamine was resuspended in 500 μL Opti-MEM, and the two mixtures were incubated separately for 5 minutes. The lipofectamine mixture was then added to the DNA mixture and incubated for 20 min. The lipofectamine/DNA complex was then added to the cell culture medium.

### Beads-Based Enrichment

Cells from a T75 flask were trypsinized and filtered through a 30 um cell strainer (Corning Falcon). Cells were centrifuged at 400 g for 4 min, then resuspended with 100 μL biotinylated isolectin B4 antibody (Enzo life sciences ALX-650-001B-MC05) and 2.4 mL cell culture medium. Cells were incubated at 4 C for 30 min on a rotator. 240 μL pre-washed Dynabeads (Invitrogen 1047) were then added to the cell/antibody mixture, and tubes were rotated at 4 C for 30 min. Tubes were placed on a magnetic rack and supernatant containing GGTA-null cells were transferred to a T25 flask containing prewarmed medium. After two days, cells were sorted by FACS.

### Single Cell Sorting

Live cells were single-cell sorted using a BD FACSAria II SORP UV (BD Biosciences) with 100 mm nozzle under sterile conditions. SSC-H versus SSC-W and FSC-H versus FSC-W doublet discrimination gates and a stringent ‘0/32/16 single-cell’ sorting mask are used to ensure that one and only one cell was sorted per well. Cells are sorted in 96-well plates with each well containing 100 μl PK15 or FF medium. After sorting, plates are centrifuged at 70 g for 3 min. Colony formation will be seen 7 days after sorting and genotyping experiments are performed 2 weeks after FACS.

### Genotyping

Genotyping of colonized porcine cells: cell cultures are dissociated using TrypLE (Invitrogen) and resuspended in PK15 medium. Cells were centrifuged at 400 g for 5 min, and medium was aspirated. Cells were resuspended in lysis solution carrying 1 μl 10× KAPA express extract buffer (KAPA Biosystems), 0.4 μl of 1 U/μl KAPA Express Extract Enzyme and 8.6 μl water. The lysis reaction is incubated at 75° C. for 15 min and inactivated the reaction at 95° C. for 5 min. All reactions are then added to 25 μl PCR reactions containing 12.5 μl 2× KAPA 2G fast (KAPA Biosystems), 100 nM PAR primers (PAR FW 5′-tgg tgg tga ccc tgt gga-3′ and PAR RV 5′-a gga aga agg aac gca aga a-3′), and 7.5 μl water. Reactions are incubated at 95° C. for 3 min followed by 45 cycles of 95° C., 20 s; 59° C., 20 s and 72° C., 20 s. PCR products were checked on EX 2% gels (Invitrogen), followed by the recovery of 300-400 bp products from the gel (QIAquick Gel Extraction Kit). PCR products were sequenced via Sanger Sequencing.

The teachings of all patents, published applications and references cited herein are incorporated by reference in their entirety.

While this invention has been particularly shown and described with references to example embodiments thereof, it will be understood by those skilled in the art that various changes in form and details may be made therein without departing from the scope of the invention encompassed by the appended claims.

What is claimed is:

1. A method of modifying a PERV-A receptor gene in a cell comprising:
introducing into the cell a nucleic acid sequence encoding a Cas9 protein and a nucleic acid sequence encoding a guide RNA,
introducing into the cell a donor nucleic acid sequence,
wherein the Cas9 protein and the guide RNA are expressed and co-localize at a genomic site near or in the PERV-A receptor gene and the donor nucleic acid sequence replaces the PERV-A receptor gene by homology directed repair (HDR).

2. The method of claim 1, wherein expression of the Cas9 protein is induced.

3. The method of claim 1, wherein the cell is from an embryo.

4. The method of claim 1, wherein the cell is a stem cell, zygote, or a germ line cell.

5. The method of claim 4, wherein the stem cell is an embryonic stem cell or pluripotent stem cell.

6. The method of claim 1, wherein the cell is a somatic cell.

7. The method of claim 6, wherein the somatic cell is a eukaryotic cell.

8. The method of claim 7, wherein the eukaryotic cell is an animal cell.

9. The method of claim 8, wherein the animal cell is a porcine cell.

10. The method of claim 9, wherein the porcine cell includes a porcine endogenous retrovirus (PERV)-free porcine fetal fibroblast cell (FF) and a PERV-free immortalized porcine kidney epithelial cell (PK).

11. The method of claim 1 further comprises modifying a second gene, GGTA1.

12. The method of claim 1, wherein the PERV-A receptor gene is a SLC52A2 gene.

13. The method of claim 1, wherein the donor nucleic acid sequence encodes a mutant SLC52A2 gene.

14. The method of claim 13, wherein the mutant SLC52A2 gene comprises a V109S, V109T, V109A or V109P substitution.

15. The method of claim 1, wherein the guide RNA is about 10 to about 1000 nucleotides.

16. The method of claim 1, wherein the guide RNA is about 15 to about 200 nucleotides.

17. A method of modifying expression of a PERV-A receptor gene in a cell comprising:
introducing into the cell a nucleic acid sequence encoding a fusion protein comprising a nuclease null Cas9 protein (dCas9) fused with a transcriptional repressor and a nucleic acid sequence encoding a guide RNA,
wherein the fusion protein and the guide RNA are expressed and co-localize at a genomic site near or in the PERV-A receptor gene and modify the expression of the PERV-A receptor gene.

18. The method of claim 17, wherein expression of the fusion protein is induced.

19. The method of claim 17, wherein the cell is from an embryo.

20. The method of claim 17, wherein the cell is a stem cell, zygote, or a germ line cell.

21. The method of claim 20, wherein the stem cell is an embryonic stem cell or pluripotent stem cell.

22. The method of claim 17, wherein the cell is a somatic cell.

23. The method of claim 22, wherein the somatic cell is a eukaryotic cell.

24. The method of claim 23, wherein the eukaryotic cell is an animal cell.

25. The method of claim 24, wherein the animal cell is a porcine cell.

26. The method of claim 25, wherein the porcine cell includes a porcine endogenous retrovirus (PERV)-free porcine fetal fibroblast cell (FF) and a PERV-free immortalized porcine kidney epithelial cell (PK).

27. The method of claim 17 wherein the PERV-A receptor gene is a SLC52A2 gene.

28. The method of claim 17, wherein the guide RNA is about 10 to about 1000 nucleotides.

29. The method of claim 17, wherein the guide RNA is about 15 to about 200 nucleotides.

30. The method of claim 17, wherein the transcription repressor comprises KRAB.

31. The method of claim 27, wherein expression of the SLC52A2 gene is repressed by dCas9-KRAB.

32. An engineered cell comprising a modified PERV-A receptor gene.

33. The engineered cell of claim 32, wherein the cell is from an embryo.

34. The engineered cell of claim 32, wherein the cell is a stem cell, zygote, or a germ line cell.

35. The engineered cell of claim 34, wherein the stem cell is an embryonic stem cell or pluripotent stem cell.

36. The engineered cell of claim 32, wherein the cell is a somatic cell.

37. The engineered cell of claim 36, wherein the somatic cell is a eukaryotic cell.

38. The engineered cell of claim 37, wherein the eukaryotic cell is an animal cell.

39. The engineered cell of claim 39, wherein the animal cell is a porcine cell.

40. The engineered cell of claim 39, wherein the porcine cell includes a porcine endogenous retrovirus (PERV)-free porcine fetal fibroblast cell (FF) and a PERV-free immortalized porcine kidney epithelial cell (PK).

41. The engineered cell of claim 32, wherein the modified PERV receptor A gene is a mutant SLC52A2 gene.

42. The engineered cell of claim 41, wherein the mutant SLC52A2 gene comprises a V109S, V109T, V109A or V109P substitution.

43. Tissues, organs or animals produced from the engineered cell of claim 32.

44. A method comprising modifying the SLC52A2 gene in a porcine cell to reduce or eliminate PERV-A binding.

45. The method of claim 44, wherein the modifying results in a V109S, V109T, V109A or V109P substitution.

46. The method of claim 44, further comprising modifying the GGTA1 gene.


SEQUENCE LISTING (6 sequences)

SEQ ID NO: 1 - DNA, 20 bp, Artificial Sequence
  Description of Artificial Sequence Synthetic oligonucleotide
gagaaaataatgaatgtcaa

SEQ ID NO: 2 - DNA, 90 bp, Artificial Sequence
  Description of Artificial Sequence Synthetic oligonucleotide
cgcagcaggccagcgccaacacaaaggtcagggccaggaaggccacagagtgactctgtcctgccatcactgtcaggtgttgccacagtg

SEQ ID NO: 3 - DNA, 90 bp, Artificial Sequence
  Description of Artificial Sequence Synthetic oligonucleotide
cgcagcaggccagcgccaacacaaaggtcagggccaggaaggccacagaagtactctgtcctgccatcactgtcaggtgttgccacagtg

SEQ ID NO: 4 - DNA, 20 bp, Artificial Sequence
  Description of Artificial Sequence Synthetic oligonucleotide
cctgacagtgatggcaggac

SEQ ID NO: 5 - DNA, 18 bp, Artificial Sequence
  Description of Artificial Sequence Synthetic primer
tggtggtgaccctgtgga

SEQ ID NO: 6 - DNA, 20 bp, Artificial Sequence
  Description of Artificial Sequence Synthetic primer
aggaagaaggaacgcaagaa